SEQUENCE LISTING (42 sequences)

SEQ ID NO: 1 - protein, 45 aa, Homo sapiens
  VARIANT 7, 8, 15, 17, 20, 21, 26, 27, 30, 33, 36, 40: Xaa = gamma carboxyglutamic acid
YNSGKLXXFVQGNLXRXCMXXKCSFXXARXVFXNTXRTTXFWKQY

SEQ ID NO: 2 - protein, 46 aa, Bos taurus
  VARIANT 7, 8, 15, 17, 20, 21, 26, 27, 30, 33, 36, 41: Xaa = gamma carboxyglutamic acid
YNSGKLXXFVQGNLXRXCMXXKCSFXXARXVFXNTXKRTTXFWKQY

SEQ ID NO: 3 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 4 - DNA, 33 bp, Artificial Sequence
  primer
ttcatattttttggaagagtttgttcaagggaa

SEQ ID NO: 5 - DNA, 28 bp, Artificial Sequence
  primer
caaaaaatatgaattatacctctttggc

SEQ ID NO: 6 - DNA, 25 bp, Artificial Sequence
  primer
ttgaattcactgaaagaacaactga

SEQ ID NO: 7 - DNA, 30 bp, Artificial Sequence
  primer
tttcagtgaattcaaaaacttctcgtgctt

SEQ ID NO: 8 - DNA, 32 bp, Artificial Sequence
  primer
gctaattcatattttttggaagagtttgttca

SEQ ID NO: 9 - DNA, 35 bp, Artificial Sequence
  primer
aaatatgaattagccctctttggccgattcagaat

SEQ ID NO: 10 - DNA, 32 bp, Artificial Sequence
  primer
gctaattcatatttattggaagagtttgttca

SEQ ID NO: 11 - DNA, 36 bp, Artificial Sequence
  primer
taaatatgaattagccctctttggccgattcagaat

SEQ ID NO: 12 - DNA, 32 bp, Artificial Sequence
  primer
gctaattcaggatttttggaagagtttgttca

SEQ ID NO: 13 - DNA, 37 bp, Artificial Sequence
  primer
aaatcctgaattagccctctttggccgattcagaatt

SEQ ID NO: 14 - DNA, 33 bp, Artificial Sequence
  primer
gaagaagcttgtagttttgaagaagcacgagaa

SEQ ID NO: 15 - DNA, 33 bp, Artificial Sequence
  primer
ctacaagcttcttccatacattctctctcaagg

SEQ ID NO: 16 - DNA, 31 bp, Artificial Sequence
  primer
gtttaagcaagggaaccttgagagagaatgt

SEQ ID NO: 17 - DNA, 34 bp, Artificial Sequence
  primer
ttcccttgcttaaactcttccaaaaaatatgaat

SEQ ID NO: 18 - DNA, 36 bp, Artificial Sequence
  primer
gctaattcaggtaaattggaagagtttgttcaaggg

SEQ ID NO: 19 - DNA, 38 bp, Artificial Sequence
  primer
tttacctgaattagccctctttggccgattcagaattt

SEQ ID NO: 20 - DNA, 33 bp, Artificial Sequence
  primer
ttcaggtcttttggaagagtttgttcaagggaa

SEQ ID NO: 21 - DNA, 33 bp, Artificial Sequence
  primer
caaaagacctgaattatacctctttggccgatt

SEQ ID NO: 22 - DNA, 38 bp, Artificial Sequence
  primer
gctaattcatataaattggaagagtttgttcaagggaa

SEQ ID NO: 23 - DNA, 38 bp, Artificial Sequence
  primer
tttatatgaattagccctctttggccgattcagaattt

SEQ ID NO: 24 - DNA, 36 bp, Artificial Sequence
  primer
taattcatataaattggaagagtttgttcaagggaa

SEQ ID NO: 25 - DNA, 34 bp, Artificial Sequence
  primer
caatttatatgaattatacctctttggccgattc

SEQ ID NO: 26 - DNA, 36 bp, Artificial Sequence
  primer
taattcatatcttttggaagagtttgttcaagggaa

SEQ ID NO: 27 - DNA, 34 bp, Artificial Sequence
  primer
caaaagatatgaattatacctctttggccgattc

SEQ ID NO: 28 - DNA, 32 bp, Artificial Sequence
  primer
gctaattcaggacttttggaagagtttgttca

SEQ ID NO: 29 - DNA, 37 bp, Artificial Sequence
  primer
aagtcctgaattagccctctttggccgattcagaatt

SEQ ID NO: 30 - DNA, 40 bp, Artificial Sequence
  primer
tgttagctggggtgaagggtgtgcaatgaaaggcaaatat

SEQ ID NO: 31 - DNA, 36 bp, Artificial Sequence
  primer
cccttcaccccagctaacaattccagttaagaaact

SEQ ID NO: 32 - DNA, 33 bp, Artificial Sequence
  primer
attaatacctacaaccatgacattgcccttctg

SEQ ID NO: 33 - DNA, 34 bp, Artificial Sequence
  primer
ggttgtaggtattaatagctgcattgtagttgtg

SEQ ID NO: 34 - DNA, 21 bp, Artificial Sequence
  primer
aaatggcactgctggttcaca

SEQ ID NO: 35 - DNA, 25 bp, Artificial Sequence
  primer
aatgggcgtggatagcggtttgact

SEQ ID NO: 36 - DNA, 24 bp, Artificial Sequence
  primer
agggatatcgacttgcagaaaacc

SEQ ID NO: 37 - DNA, 23 bp, Artificial Sequence
  primer
tgctgacaaggaatacacgaaca

SEQ ID NO: 38 - DNA, 24 bp, Artificial Sequence
  primer
ctttccgcctcagaagccatagag

SEQ ID NO: 39 - DNA, 22 bp, Artificial Sequence
  primer
tggcaactagaaggcacagtcg

SEQ ID NO: 40 - DNA, 24 bp, Artificial Sequence
  primer
agactcttccccagccacttacat

SEQ ID NO: 41 - DNA, 23 bp, Artificial Sequence
  primer
aaatggcactgctggttcacagg

SEQ ID NO: 42 - DNA, 20 bp, Artificial Sequence
  primer
taatacgactcactataggg

(19) United States
(12) Patent Application Publication
(10) Pub. No.: US 2015/0337284 A1
(43) Pub. Date: Nov. 26, 2015

(54) FACTOR IX VARIANTS

(71) Applicant: REGENTS OF THE UNIVERSITY OF MINNESOTA, St. Paul, MN (US)

(72) Inventors: Gary L. Nelsestuen, St. Paul, MN (US); Stephen Barrett Harvey, Minneapolis, MN (US)

(21) Appl. No.: 14/439,454

(22) PCT Filed: Sep. 30, 2013
(86) PCT No.: PCT/US2013/062578

§ 371 Date: Apr. 29, 2015

Related U.S. Application Data
(60) Provisional application No. 61/719,576, filed on Oct. 29, 2012.

(51) Int. Cl. C12N 9/64; A61K 38/48

(52) U.S. Cl. CPC C12N 9/644; A61K 38/4846

(57) ABSTRACT

Variants of factor IX with increased membrane binding affinity, and the use of such variants for treating factor IX deficiency, are described.

### CROSS-REFERENCE TO RELATED APPLICATIONS

This application claims priority from U.S. Provisional Application Ser. No. 61/719,576, filed Oct. 29, 2012.

### TECHNICAL FIELD

This disclosure relates to variants of blood clotting factor IX that have increased affinity for biological membranes, and the use of such variants for treating factor IX deficiency or factor VIII deficiency with inhibitors.

### BACKGROUND

Factor IX is commonly used as a therapeutic agent for people with hemophilia B, the genetic deficiency of factor IX, and in hemophilia A patients who develop inhibitors to factor VIII replacement therapy. Protein replacement therapy is the primary treatment.

### SUMMARY

This disclosure is based in part on the identification of factor IX variants that have increased affinity for biological membranes. These variants may be used for enzyme replacement therapy in factor IX deficiency, and they also may be useful as bypass agents that can function in the absence of factor VIII. As described herein, factor IX variants with increased membrane binding affinity can have advantages over other approaches such as increased function at the active site.

In one aspect, this disclosure features a variant factor IX or factor IXa polypeptide having enhanced membrane binding affinity relative to a corresponding native factor IX or factor IXa polypeptide, wherein the polypeptide comprises a modified Gla domain with an amino acid substitution at one or more of positions 1, 4, and 5 as compared to the sequence set forth in SEQ ID NO:1 or SEQ ID NO:2. The Gla domain can comprise an amino acid substitution at position 1 (e.g., an alanine residue substituted at position 1). The Gla domain can comprise an amino acid substitution at position 4 (e.g., a tyrosine residue substituted at position 4). The Gla domain can comprise an amino acid substitution at position 5 (e.g., a leucine residue substituted at position 5). The Gla domain can comprise amino acid substitutions at positions 1 and 4 (e.g., an alanine residue substituted at position 1 and a tyrosine residue substituted at position 4). The Gla domain can comprise amino acid substitutions at positions 1, 4, and 5 (e.g., an alanine residue substituted at position 1, a tyrosine residue substituted at position 4, and a leucine residue substituted at position 5). The variant factor IX or factor IXa polypeptide further can have one or more amino acid substitutions at positions outside the Gla domain. For example, the variant factor IX or factor IXa polypeptide further can have an amino acid substitution at one or more of positions 86 (e.g., a threonine residue substituted at position 86), 181 (e.g., an isoleucine residue substituted at position 181), 259 (e.g., a phenylalanine residue substituted at position 259), 265 (e.g., a threonine residue substituted at position 265, 277 (e.g., an alanine residue substituted at position 277), 338 (e.g., an aspartic acid residue substituted at position 338), 345 (e.g., a phenylalanine residue substituted at position 345), 383 (e.g., a valine residue substituted at position 383), and/or 388 (e.g., a glycine residue substituted at position 388). In some embodiments, the variant factor IX or factor IXa polypeptide can have substitutions at positions 1, 4, and 5 of the Gla domain (e.g., an alanine residue substituted at position 1, a tyrosine residue substituted at position 4, and a leucine residue substituted at position 5 of the Gla domain), and at positions 265, 383, and 388 (e.g., a threonine residue substituted at position 265, a valine residue substituted at position 383, and a glycine residue substituted at position 388.

In another aspect, this disclosure features a pharmaceutical composition comprising a pharmaceutically acceptable carrier and an amount of a variant factor IX or factor IXa polypeptide effective to increase clot formation in a mammal, wherein the polypeptide comprises a modified Gla domain with an amino acid substitution at one or more of positions 1, 4, or 5 as compared to the sequence set forth in SEQ ID NO:1 or SEQ ID NO:2. The polypeptide can comprise a modified Gla domain with amino acid substitutions at positions 1, 4, and 5 (e.g., an alanine residue substituted at position 1, a tyrosine residue substituted at position 4, and a leucine residue substituted at position 5).

In another aspect, this disclosure features a mammalian host cell that expresses a variant factor IX or factor IXa polypeptide having enhanced membrane binding affinity relative to a corresponding native factor IX or factor IXa polypeptide, wherein the polypeptide comprises a modified Gla domain with an amino acid substitution at one or more of positions 1, 4, or 5 as compared to the sequence set forth in SEQ ID NO:1 or SEQ ID NO:2. The polypeptide can comprise a modified Gla domain with amino acid substitutions at positions 1, 4, and 5 (e.g., an alanine residue substituted at position 1, a tyrosine residue substituted at position 4, and a leucine residue substituted at position 5).

In still another aspect, this disclosure features a method of increasing clot formation in a mammal, comprising administering to the mammal an amount of a variant factor IX or factor IXa polypeptide effective to increase clot formation in the mammal, wherein the polypeptide comprises a modified Gla domain with an amino acid substitution at one or more of positions 1, 4, or 5 as compared to the sequence set forth in SEQ ID NO:1 or SEQ ID NO:2. The polypeptide can comprise a modified Gla domain with amino acid substitutions at positions 1, 4, and 5 (e.g., an alanine residue substituted at position 1, a tyrosine residue substituted at position 4, and a leucine residue substituted at position 5).

In yet another aspect, this disclosure features an isolated nucleic acid comprising a nucleic acid sequence encoding a variant factor IX or factor IXa polypeptide having enhanced membrane binding affinity relative to a corresponding native factor IX or factor IXa polypeptide, wherein the polypeptide comprises a modified Gla domain with an amino acid substitution at one or more of positions 1, 4, or 5 as compared to the sequence set forth in SEQ ID NO:1 or SEQ ID NO:2. The polypeptide can comprise a modified Gla domain with amino acid substitutions at positions 1, 4, and 5 (e.g., an alanine residue substituted at position 1, a tyrosine residue substituted at position 4, and a leucine residue substituted at position 5).

This disclosure also features a method for producing a variant factor IX or factor IXa polypeptide having enhanced membrane binding affinity relative to a corresponding native factor IX or factor IXa polypeptide, wherein the polypeptide comprises a modified Gla domain with an amino acid substitution at one or more of positions 1, 4, or 5 as compared to the sequence set forth in SEQ ID NO:1 or SEQ ID NO:2. The method can include: (a) providing a culture of mammalian host cells as described herein under conditions that permit expression of the polypeptide, and (b) recovering the polypeptide. The polypeptide can comprise a modified Gla domain with amino acid substitutions at positions 1, 4, and 5 (e.g., an alanine residue substituted at position 1, a tyrosine residue substituted at position 4, and a leucine residue substituted at position 5).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph plotting membrane binding isotherms for factor IX variant proteins. M2/M1 is the ratio of molecular weight of the protein-membrane complex to that of the membrane particle alone. The dashed line is the theoretical value if all of the added protein bound to the membrane. Titrations were carried out in 0.05 M Tris buffer, 0.1 M NaCl, 5 mM CaCl₂. Open diamonds=wild type (WT) recombinant factor IX, open squares=the triple mutant Y1A/G4Y/KSL, open triangles=Y1A variant, solid triangles=G4Y variant, open circles=Y1A/G4Y variant. Error bars represent the SD for three measurements.

FIG. 2 is a graph plotting blood clotting as a function of recombinant factor IXa or VIIa concentration in factor IX-deficient plasma. Tris buffer (0.1 mL, 0.05 M, pH 7.35) containing 0.1 M NaCl, 100 μg/mL of phospholipid vesicles (PS/PC 25/75), and 7.5 mM CaCl₂ was mixed with 50 μL of factor IX-deficient plasma and the factor concentrations shown. Clotting times were measured by the hand tilt method. The proteins used were recombinant triple mutant Y1A/G4Y/K5L factor IXa (open squares), recombinant WT factor IXa (open circles), triple mutant in plasma deficient in both IX and VIII (open triangles), triple mutant in factor VIII-deficient plasma (X), recombinant WT IXa in factor IX and VIII-deficient plasma (open diamonds), and recombinant factor VIIa (NOVOSEVEN®; +). The equations were for the best fit linear relationship for the data.

FIG. 3 is a graph plotting whole blood clotting as a function of factor IXa concentration in factor VIII-deficient blood. Blood was collected in 0.1 M sodium citrate (9:1, v/v). Factor VIII was inactivated by addition of 8 μg of anti-factor VIII/mL. To start coagulation, 0.1 mL of blood was mixed with 2.4 μL of 0.4 M CaCl₂ and the appropriate amount of protein. Approximately 50 μL was added to the ACT-LR cuvette in the Hemochron Junior Signature Micro blood clotting apparatus, which measured the time required for the blood to coagulate. The proteins titrated included the Y1A/G4Y/K5L factor IXa mutant (open circles), WT factor IXa (squares), Y1A/G4Y/K5L mutant factor IX zymogen (X), and factor VIIa (solid triangles). WT factor IX zymogen (1 μM) did not cause blood to clot within the 400 second cutoff for the instrument. Error bars represent the SD of 4 measurements.

FIG. 4 is a graph plotting whole blood clotting activity of the Gla domain triple mutant (Y1A/G4Y/K5L, diamonds), as well as a protein containing three additional changes (K265T/I383V/E388G, squares).

### DETAILED DESCRIPTION

Vitamin K-dependent proteins are involved in a number of biological processes, including blood coagulation (see, e.g., Furie and Furie, 1988, Cell, 53:505-518). Factor IX is an example of a vitamin K-dependent protein. Like other vitamin K-dependent proteins, factor IX contains a domain that is about 45 amino acid residues in length and contains 9 to 13 gamma-carboxyglutamic acid (Gla) residues. The Gla residues are needed for proper calcium binding and membrane interaction, and are produced by enzymes in the liver that utilize vitamin K to carboxylate the side chains of glutamic acid residues in protein precursors. Although the Gla-containing regions (also referred to herein as the “Gla domain”) of vitamin K-dependent proteins (which also include protein Z, protein S, prothrombin, factor X, protein C, and factor VII) have a high degree of sequence homology, they have at least a 1000-fold range in membrane affinity (McDonald et al., Biochemistry, 1997, 36:5120-5137).

Factor IX is produced in the liver as a zymogen or inactive enzyme precursor. It is processed in the liver to remove an amino terminal signal peptide, glycosylated, carboxylated, and otherwise modified before transport into the blood stream. The zymogen is activated through proteolytic cleavage by factor XIa or factor VIIa to produce a two-chain form (factor IXa) in which the chains are linked by disulfide bridging. When activated in the presence of Ca2+, membrane phospholipids, and an activated Factor VIII cofactor, factor IXa hydrolyses an arginine-isoleucine bond in factor X to form factor Xa.

As described herein, factor IX variants with increased affinity for biological membranes have now been identified. It is to be understood that the variants described herein are with reference to both factor IX and factor IXa. In other words, a “variant factor IX polypeptide” as well as a “factor IX variant” refers to a factor IX polypeptide or a factor IXa polypeptide that contains mutations as described herein.

The variant factor IX polypeptides described herein may be used for enzyme replacement therapy in factor IX deficiency (e.g., hemophilia B). In some embodiments, the variant factor IX polypeptides can be useful as bypass agents that can function in the absence of factor VIII (e.g., in hemophilia A patients who develop inhibitors to factor VIII replacement therapy). The total market for bypass reagents may be larger than that for hemophilia B. The variant factor IX polypeptides provided herein differ from previously used factor IX therapeutics in that they have increased function due to enhanced membrane affinity. As compared to other factor IX therapeutic proteins, the variants described herein may be used at a lower dosage. In some embodiments, the factor IX variants can be coupled with other technologies, such as PEG attachment, additional glycosylation or in a manner to generate Fc fusion proteins to produce even better combination characteristics. Normally, PEG or other large attachments to a polypeptide tend to lower protein function. The modifications described herein may replace the lost function to produce a superior outcome overall.

This disclosure features isolated variant factor IX polypeptides. An “isolated polypeptide” has been separated from cellular components that naturally accompany it. Typically, the polypeptide is isolated when it is at least 60% (e.g., 70%, 80%, 90%, or 95%), by weight, free from proteins and naturally-occurring organic molecules that are naturally associated with it. As used herein, the term “polypeptide” is any chain of at least five amino acids that retains the ability to bind cofactors or membranes. Amino acids are designated herein by standard three-letter and one-letter abbreviations.

The variant factor IX polypeptides provided herein contain a Gla domain that can have an amino acid sequence with one or more (e.g., one, two, three, four, five, or more than five) amino acid substitutions as compared with the Gla domain of a corresponding wild type factor IX polypeptide. A representative sequence of a wild type human factor IX Gla domain is YNSGKLXXFVQGNLXRXCMXXKCSFXXARXVFXNTXRTTXF WKQY (SEQ ID NO:1), where “X” represents Gla. A representative sequence of a wild type bovine factor IX Gla domain is YNSGKLXXFVQGNLXRXCMXXKCSFXXA RXVFXNTXKTTXFWKQY (SEQ ID NO:2), where “X” represents Gla. A representative full length sequence for human factor IX is set forth in the Swiss-Prot database under accession no. P00740.2:
MQRVNMIMAESPGLITICLLGYLLSAECTVFLDHENANKILNRPKRY NSGKLEEFVQGNLERECMEEKCSFEEAREVFENTERTTEFWKQYVD GDQCESNPCLNGGSCKDDINSYECWCPFGFEGKNCELDVTCNIKNGR CEQFCKNSADNKVVCSCTEGYRLAENQKSCEPAVPFPCGRVSVSQTS KLTRAETVFPDVDYVNSTEAETILDNITQSTQSFNDFTRVVGGEDAKP GQFPWQVVLNGKVDAFCGGSIVNEKWIVTAAHCVETGVKITVVAGE HNIEETEHTEQKRNVIRIIPHHNYNAAINKYNHDIALLELDEPLVLNSY VTPICIADKEYTNIFLKFGSGYVSGWGRVFHKGRSALVLQYLRVPLVD RATCLRSTKFTIYNNMFCAGFHEGGRDSCQGDSGGPHVTEVEGTSFL TGIISWGEECAMKGKYGIYTKVSRYVNWIKEKTKLT (SEQ ID NO:3)

The substitutions within the factor IX polypeptide may be conservative or non-conservative. Conservative amino acid substitutions are those that replace an amino acid with another amino acid of the same class, whereas non-conservative amino acid substitutions are those that replace an amino acid with an amino acid of a different class. Non-conservative substitutions may result in a substantial change in the hydrophobicity of the polypeptide or in the bulk of a residue side chain. In addition, non-conservative substitutions may make a substantial change in the charge of the polypeptide, such as reducing electropositive charges or introducing electronegative charges. Examples of non-conservative substitutions include a basic amino acid for a non-polar amino acid, or a polar amino acid for an acidic amino acid.

In some embodiments, a variant factor IX can have amino acid substitutions at one or more of positions 1, 4, and 5 of the Gla domain. Thus, a variant factor IX polypeptide can have a substitution at position 1 of the Gla domain, a substitution at position 4 of the Gla domain, a substitution at position 5 of the Gla domain, substitutions at positions 1 and 4 of the Gla domain, substitutions at positions 1 and 5 of the Gla domain, substitutions at positions, 4 and 5 of the Gla domain, or substitutions at positions 1, 4, and 5 of the Gla domain. For example, a variant factor IX polypeptide can have an Ala substituted for the Tyr at position 1 of the Gla domain, a Tyr substituted for the Gly at position 4 of the Gla domain, a Leu substituted for the Lys at position 5 of the Gla domain, or any combination thereof.

Factor IX variants with increased membrane binding affinity can have advantages over other types of factor IX variants, such as those with increased function at the active site. For example, an important aspect in one concept for factor IXa action is the need to diffuse through solution from tissue factor- (TF-) bearing cells where the zymogen, factor IX, is activated to the platelet plug where it then combines with factor VIIIa to generate the enzyme that activates factor X to Xa. Transfer is feasible due to the low activity of the factor IXa active site in the absence of factor VIIIa. Active sites that are fully developed may be rapidly inhibited by abundant protease inhibitors of the blood. Proteins that have increased membrane affinity but contain the native active site typically will retain this necessary flexibility of the coagulation system.

Factor IX zymogen variants may be able to bypass factor VIII by having both enhanced activation and subsequent activity toward factor X. Factor IX polypeptides with enhanced membrane binding ability, as described herein, showed an increase in factor VIII-independent action in blood clotting assays. Thus, these zymogens may be useful for bypass activity for persons with hemophilia A and inhibitors. That is, increased membrane affinity can enhance activation rates of factor IX and target factor IXa to the platelet plug without increasing the rate of its inhibition. Use of factor IXa or factor IX as a bypass agent is likely independent of its association with collagen in the sub-endothelium. Consequently, the full range of mutations described herein can be used.

In other embodiments, the factor IX variants described herein can include additional mutations outside the Gla domain. These may include variations that otherwise enhance factor IX activity in the presence or absence of factor VIIIa or prolong circulation lifetime or provide other advantage. For example, a variant with three changes (V86T/E277A/R338D) had enhanced function in some assays but not others (Lin et al., J. Thromb. Haemostasis, 8:1773-1783, 2010), possibly due to rapid neutralization of the enzyme with higher activity by inhibitors of factor IXa in plasma. In another example, five mutations (Y259F/K265T/Y345F/I383V/E388G) were identified that enhanced association with factor X and the activity of the active site (Hartman et al., J. Thromb. Haemostatsis, 7:1656-1662, 2009). Another group used three variations (V181I/K265T/I383V) to generate increased activity, especially in the absence of factor VIII, where activity was 6% the activity observed in the presence of factor VIII (Milanov et al., Blood, 119:602-611, 2012). Combining such variants or others with those that increase membrane affinity as described herein may provide even greater function in the absence of factor VIIIa. For example, a variant factor IX polypeptide can contain mutations at one or more of positions 1, 4, and 5 of the Gla domain as set forth herein, in combination with amino acid substitutions at one or more of positions 86 (e.g., a threonine residue substituted at position 86), 181 (e.g., an isoleucine residue substituted at position 181), 259 (e.g., a phenylalanine residue substituted at position 259), 265 (e.g., a threonine residue substituted at position 265, 277 (e.g., an alanine residue substituted at position 277), 338 (e.g., an aspartic acid residue substituted at position 338), 345 (e.g., a phenylalanine residue substituted at position 345), 383 (e.g., a valine residue substituted at position 383), and/or 388 (e.g., a glycine residue substituted at position 388). In some embodiments, for example, a factor IX variant can have substitutions at positions 1, 4, and 5 of the Gla domain (e.g., an alanine residue substituted at position 1, a tyrosine residue substituted at position 4, and a leucine residue substituted at position 5 of the Gla domain), and at positions 265, 383, and 388 (e.g., a threonine residue substituted at position 265, a valine residue substituted at position 383, and a glycine residue substituted at position 388

In some embodiments, the factor IX variants described herein can be combined with protein modifications that increase circulation lifetime. For example, variant proteins can be modified with polyethylene glycol (PEG), additional glycosylation sites, polysialylation, or Fc fusion proteins. Such changes can reduce function of the modified protein (Stone et al., Biochemistry, 41:15820-15825, 2002), but increased function by enhanced membrane affinity can offset this loss of function, and can provide proteins with increased or full function as well as an increased circulation lifetime. See, e.g., the presentation on hemophilia research that is available online at press.bayerhealthcare.com/html/pdf/presse/en/electronic_press_kits/Hematology_Conference/Hematology_Conf erence_Preclinical_Research_Jesper_Haaning.pdf).

In another example, a factor IX variant can include an inactivated cleavage site, such that the polypeptide is not converted to an active form. Thus, a factor IX variant containing an inactivated cleavage site would not be converted to factor IXa. In general, an Arg residue is found at the cleavage site of vitamin K-dependent polypeptides. Any residue can be substituted for the Arg at this position to inactivate the cleavage site. In particular, an Ala residue could be substituted for the Arg at amino acid 191 or 226 of factor IX. Factor IX variants that further contain an inactivated cleavage site can act as inhibitors.

Host cells (e.g., mammalian host cells) containing variant factor IX polypeptides with a modified Gla domain that enhances membrane-binding affinity also are provided herein. Suitable host cells are able to modify Glu residues within factor IX variants to Gla residues. Mammalian cells derived from kidney and liver can be especially useful as host cells.

Isolated nucleic acids encoding variant factor IX polypeptides also are provided herein. Such nucleic acids can be produced by standard techniques. As used herein, “isolated” refers to a sequence corresponding to part or all of a gene encoding a variant factor IX polypeptide, but free of sequences that normally flank one or both sides of the wild-type gene in a mammalian genome. An isolated polynucleotide can be, for example, a recombinant DNA molecule, provided that one of the nucleic acid sequences normally found immediately flanking that recombinant DNA molecule in a naturally-occurring genome is removed or absent. Thus, isolated polynucleotides include, without limitation, a DNA that exists as a separate molecule (e.g., a cDNA or genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated polynucleotide can include a recombinant DNA molecule that is part of a hybrid or fusion polynucleotide.

It will be apparent to those of skill in the art that a polynucleotide existing among hundreds to millions of other polynucleotides within, for example, cDNA or genomic libraries, or gel slices containing a genomic DNA restriction digest, is not to be considered an isolated polynucleotide.

Isolated nucleic acids can be at least about 15 nucleotides in length. For example, an isolated nucleic acid can be at least 15, at least 20, at least 50, at least 100, or at least 150 nucleotides in length, or any range there between (e.g., 15 to 20, 20 to 50, 50 to 100, 20 to 100, or 50 to 150 nucleotides in length). In some embodiments, an isolated nucleic acid can encode a full-length factor IX variant. Nucleic acid molecules can be DNA or RNA, linear or circular, and in sense or antisense orientation.

Specific point changes can be introduced into a nucleic acid sequence encoding a wild-type factor IX polypeptide using methods that are known in the art. For example, oligonucleotide-directed mutagenesis can be used to generate nucleic acids encoding factor IX variants. In this method, a desired change is incorporated into an oligonucleotide, which then is hybridized to the wild-type nucleic acid. The oligonucleotide is extended with a DNA polymerase, creating a heteroduplex that contains a mismatch at the introduced point change, and a single-stranded nick at the 5′ end, which is sealed by a DNA ligase. The mismatch is repaired upon transformation of E. coli or other appropriate organism, and the gene encoding the modified factor IX polypeptide can be re-isolated from E. coli or other appropriate organism. Kits for introducing site-directed mutations can be purchased commercially. For example, Muta-Gene7 in-vitro mutagenesis kits can be purchased from Bio-Rad Laboratories, Inc. (Hercules, Calif.).

Polymerase chain reaction (PCR) techniques also can be used to introduce mutations. See, for example, Vallette et al., Nucleic Acids Res., 1989, 17(2):723-733. PCR refers to a procedure or technique in which target nucleic acids are amplified. Sequence information from the ends of the region of interest or beyond typically is employed to design oligonucleotide primers that are identical in sequence to opposite strands of the template to be amplified, whereas for introduction of mutations, oligonucleotides that incorporate the desired change are used to amplify the nucleic acid sequence of interest. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995.

Nucleic acids encoding variant factor IX polypeptides also can be produced by chemical synthesis, either as a single nucleic acid molecule or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a double-stranded nucleic acid molecule per oligonucleotide pair, which then can be ligated into a vector.

Variant factor IX polypeptides can be produced by ligating a nucleic acid sequence encoding the polypeptide into a nucleic acid construct such as an expression vector, and transforming a bacterial or eukaryotic host cell with the expression vector. In general, nucleic acid constructs include a regulatory sequence operably linked to a nucleic acid sequence encoding a variant factor IX polypeptide. Regulatory sequences do not typically encode a gene product, but instead affect the expression of the nucleic acid sequence. As used herein, “operably linked” refers to connection of the regulatory sequences to the nucleic acid sequence in such a way as to permit expression of the nucleic acid sequence. Regulatory elements can include, for example, promoter sequences, enhancer sequences, response elements, or inducible elements.

In bacterial systems, a strain of E. coli such as BL-21 can be used. Suitable E. coli vectors include, without limitation, the pGEX series of vectors that produce fusion proteins with glutathione S-transferase (GST). Transformed E. coli typically are grown exponentially then stimulated with isopropylthiogalactopyranoside (IPTG) prior to harvesting. In general, such fusion proteins are soluble and can be purified easily from lysed cells by adsorption to glutathione-agarose beads followed by elution in the presence of free glutathione. The pGEX vectors are designed to include thrombin or factor Xa protease cleavage sites such that the cloned target gene product can be released from the GST moiety.

In eukaryotic host cells, a number of viral-based expression systems can be utilized to express variant factor IX polypeptides. A nucleic acid encoding a variant factor IX polypeptide can be cloned into, for example, a baculoviral vector such as pBlueBac (Invitrogen, Carlsbad, Calif.) and then used to co-transfect insect cells such as Spodoptera frugiperda (Sf9) cells with wild-type DNA from Autographa californica multiply enveloped nuclear polyhedrosis virus (AcMNPV). Recombinant viruses producing the variant factor IX polypeptides can be identified by standard methodology. Alternatively, a nucleic acid encoding a factor IX variant can be introduced into a SV40, retroviral, or vaccinia based viral vector and used to infect suitable host cells.

Mammalian cell lines that stably express variant factor IX polypeptides can be produced using expression vectors with the appropriate control elements and a selectable marker. For example, the eukaryotic expression vector pCDNA.3.1+(Invitrogen) is suitable for expression of variant factor IX polypeptides in, for example, COS cells, HEK293 cells, or baby hamster kidney cells. Following introduction of the expression vector by electroporation, DEAE dextran-, calcium phosphate-, liposome-mediated transfection, or other suitable method, stable cell lines can be selected. Alternatively, transiently transfected cell lines are used to produce variant factor IX polypeptides. Variant factor IX polypeptides also can be transcribed and translated in vitro using wheat germ extract or rabbit reticulocyte lysate.

The variant factor IX polypeptides provided herein can be purified from conditioned cell medium by applying the medium to an immunoaffinity column. For example, an antibody having specific binding affinity for factor IX can be used to purify variant factor IX. Alternatively, concanavalin A (Con A) chromatography and anion-exchange chromatography (e.g., DEAE) can be used in conjunction with affinity chromatography to purify variant factor IX. Calcium dependent or independent monoclonal antibodies that have specific binding affinity for factor IX can be used in the purification procedure.

Variant factor IX polypeptides also can be chemically synthesized using standard techniques. See, e.g., Muir and Kent, Curr. Opin. Biotechnol., 1993, 4(4):420-427, for a review of protein synthesis techniques.

This disclosure also provides pharmaceutical compositions containing a pharmaceutically acceptable carrier and variant factor IX polypeptide as described herein, in an amount effective to increase clot formation in a mammal. The concentration of a variant factor IX polypeptide effective to increase clot formation may vary, depending on a number of factors, including the preferred dosage of the compound to be administered, the chemical characteristics of the compounds employed, the formulation of the compound excipients and the route of administration. The optimal dosage of a pharmaceutical composition to be administered may also depend on such variables as the overall health status of the particular patient and the relative biological efficacy of the compound selected. These pharmaceutical compositions may be used to regulate coagulation in vivo. For example, the compositions may be used generally for the treatment of hemophilia (e.g., hemophilia A or hemophilia B). Altering only a few amino acid residues of the polypeptide as described herein, generally does not significantly affect the antigenicity of the mutant polypeptides.

Factor IX variants as described herein can be formulated into pharmaceutical compositions by admixture with pharmaceutically acceptable, non-toxic excipients or carriers. Such compounds and compositions can be prepared for parenteral administration (e.g., in the form of liquid solutions or suspensions in aqueous physiological buffer solutions), for oral administration (e.g., in the form of tablets or capsules), or for intranasal administration (e.g., in the form of powders, nasal drops, or aerosols). Compositions for other routes of administration can be prepared as desired using standard methods.

Formulations for parenteral administration may contain as common excipients sterile water or saline, polyalkylene glycols such as polyethylene glycol, oils of vegetable origin, hydrogenated naphthalenes, and the like. In particular, biocompatible, biodegradable lactide polymer, lactide/glycolide copolymer, or polyoxethylene-polyoxypropylene copolymers are examples of excipients for controlling the release of a compound of the invention in vivo. Other suitable parenteral delivery systems include ethylene-vinyl acetate copolymer particles, osmotic pumps, implantable infusion systems, and liposomes. Formulations for inhalation administration may contain excipients such as lactose, if desired. Inhalation formulations may be aqueous solutions containing, for example, polyoxyethylene-9-lauryl ether, glycocholate and deoxycholate, or they may be oily solutions for administration in the form of nasal drops. If desired, the compounds can be formulated as gels to be applied intranasally. Formulations for parenteral administration may also include glycocholate for buccal administration. In some embodiments, a pharmaceutical composition also can contain soluble tissue factor.

Methods for increasing clot formation in a mammal also are provided herein. The methods can include administering an amount of a variant factor IX polypeptide effective to increase clot formation in the mammal. The variant factor IX polypeptide can have a Gla domain with amino acid substitutions that enhances membrane-binding affinity of the polypeptide relative to a corresponding wild type factor IX polypeptide, as described herein. In some embodiments, activity is additionally enhanced by amino acid substitutions outside of the Gla domain.

Articles of manufacture also are provided herein. The articles of manufacture can contain, for example, variant factor IX polypeptides with increased membrane binding affinity as described herein, nucleic acid molecules encoding such variant factor IX polypeptides, compositions containing such nucleic acid molecules or polypeptides, or cell lines containing such nucleic acid molecules or polypeptides. The items included in the articles of manufacture can be used, for example, as research tools, or therapeutically.

The invention will be further described in the following examples, which do not limit the scope of the invention described in the claims.

### EXAMPLES

### Example 1

### Production of Primers and Incorporation of Plasmids into K293 Cells

Amino acid codon changes in recombinant human factor IX were made by PCR-based site-directed mutagenesis. Briefly, primers were synthesized with the desired codon changes and used in PCR to amplify two overlapping fragments. The two fragments were gel-purified and used as (overlapping) templates in a second PCR step primed by flanking PCR primers. Appropriate restriction enzymes were used to cleave and subclone the PCR fragments into the corresponding sites in the Factor IX expression plasmid, pRC-Fa9, using standard procedures (e.g., Current Protocols in Molecular Biology, Vol. 1-4, Ausubel et al., ed., John Wiley and Sons, USA, updates to 2010). The primers shown in Table 1 were used in appropriate order and combination to generate DNA sequences encoding the protein variants described below. The inserts in the resulting clones were completely sequenced across the cloned segments. Plasmids were introduced into human HEK293 cells (Gibco 293-H) by lipofection.

TABLE 1
Primers used for PCR-based site-directed mutagenesis

| | | |
|---|---|---|
| Primer | | SEQ ID |
| name | Sequence | NO: |

M1 primers

| | | |
|---|---|---|
| P182SM1 | TTCATATTTTTTGGAAGAGTTTGTTCAAGGGAA | 4 |
| P182-AM1 | CAAAAAATATGAATTATACCTCTTTGGC | 5 |

M2 primers

| | | |
|---|---|---|
| P182-SM2 | TTGAATTCACTGAAAGAACAACTGA | 6 |
| P182-AM2 | TTTCAGTGAATTCAAAAACTTCTCGTGCTT | 7 |

M3 primers

| | | |
|---|---|---|
| P182-SM3 | GCTAATTCATATTTTTTGGAAGAGTTTGTTCA | 8 |
| P182-AM3 | AAATATGAATTAGCCCTCTTTGGCCGATTCAGAAT | 9 |

M4 primers

| | | |
|---|---|---|
| P182-SM4 | GCTAATTCATATTTATTGGAAGAGTTTGTTCA | 10 |
| P182-AM4 | TAAATATGAATTAGCCCTCTTTGGCCGATTCAGAAT | 11 |

M5 primers

| | | |
|---|---|---|
| P182-SM5 | GCTAATTCAGGATTTTTGGAAGAGTTTGTTCA | 12 |
| P182-AM5 | AAATCCTGAATTAGCCCTCTTTGGCCGATTCAGAATT | 13 |

M6 primers

| | | |
|---|---|---|
| P182-SM6 | GAAGAAGCTTGTAGTTTTGAAGAAGCACGAGAA | 14 |
| P182-AM6 | CTACAAGCTTCTTCCATACATTCTCTCTCAAGG | 15 |

M7 primers

| | | |
|---|---|---|
| P182-SM7 | GTTTAAGCAAGGGAACCTTGAGAGAGAATGT | 16 |
| P182-AM7 | TTCCCTTGCTTAAACTCTTCCAAAAAATATGAAT | 17 |

M8 primers

| | | |
|---|---|---|
| P182-SM8 | GCTAATTCAGGTAAATTGGAAGAGTTTGTTCAAGGG | 18 |
| P182-AM8 | TTTACCTGAATTAGCCCTCTTTGGCCGATTCAGAATTT | 19 |

M9 primers

| | | |
|---|---|---|
| P182-SM9 | TTCAGGTCTTTTGGAAGAGTTTGTTCAAGGGAA | 20 |
| P182-AM9 | CAAAAGACCTGAATTATACCTCTTTGGCCGATT | 21 |

M10 primers

| | | |
|---|---|---|
| P182-SM10 | GCTAATTCATATAAATTGGAAGAGTTTGTTCAAGGGAA | 22 |
| P182-AM10 | TTTATATGAATTAGCCCTCTTTGGCCGATTCAGAATTT | 23 |

M11 primers

| | | |
|---|---|---|
| P182-SM11 | TAATTCATATAAATTGGAAGAGTTTGTTCAAGGGAA | 24 |
| P182-AM11 | CAATTTATATGAATTATACCTCTTTGGCCGATTC | 25 |

M12 primers

| | | |
|---|---|---|
| P182-SM12 | TAATTCATATCTTTTGGAAGAGTTTGTTCAAGGGAA | 26 |
| P182-AM12 | CAAAAGATATGAATTATACCTCTTTGGCCGATTC | 27 |

M13 primers

| | | |
|---|---|---|
| P182-SM13 | GCTAATTCAGGACTTTTGGAAGAGTTTGTTCA | 28 |
| P182-AM13 | AAGTCCTGAATTAGCCCTCTTTGGCCGATTCAGAATT | 29 |

M15 primers

| | | |
|---|---|---|
| P182-SM15 | TGTTAGCTGGGGTGAAGGGTGTGCAATGAAAGGCAAATAT | 30 |
| P182-AM15 | CCCTTCACCCCAGCTAACAATTCCAGTTAAGAAACT | 31 |
| P182-SM16 | ATTAATACCTACAACCATGACATTGCCCTTCTG | 32 |
| P182-AM16 | GGTTGTAGGTATTAATAGCTGCATTGTAGTTGTG | 33 |

Additional primers used for PCR and DNA sequencing

| | | |
|---|---|---|
| P182-A528 | AAATGGCACTGCTGGTTCACA | 34 |
| P130-S639 | AATGGGCGTGGATAGCGGTTTGACT | 35 |
| P182-S1374 | AGGGATATCGACTTGCAGAAAACC | 36 |
| P182-S1906 | TGCTGACAAGGAATACACGAACA | 37 |
| P182-A2660 | CTTTCCGCCTCAGAAGCCATAGAG | 38 |
| P182-A2438 | TGGCAACTAGAAGGCACAGTCG | 39 |
| P182-A1976 | AGACTCTTCCCCAGCCACTTACAT | 40 |
| P182-A1426 | AAATGGCACTGCTGGTTCACAGG | 41 |
| T7-Seq2 | TAATACGACTCACTATAGGG | 42 |

### Example 2

### Tissue Culture, Growth and Expression of Factor IX Proteins

Human embryonic kidney cells 293-H (Gibco 11631-017) were maintained in Dulbecco's modified Eagle's medium containing 1.0 mM non-essential amino acids, 50 units/ml penicillin, 50 ng/ml streptomycin, 10 ng/ml Vitamin K1 and 5% fetal bovine serum. Transfections were carried out by lipofection, using Geneticin selection. Transfected cell lines were maintained in the same medium containing 200 μg/ml Geneticin (Life Technologies). The 293 cells that had been transfected with appropriate plasmids were chosen for factor IX production by screening culture supernatants for the presence of factor IX using immunochemical dot-blot analysis. High producing strains were acclimated to serum-free medium (SFM II, (Gibco)), scaled up and used to seed 1-Liter CELLine CL 1000 culture flasks (Wilson Wolf Mfg. Corp.) in which the cell chamber contained 15-19 ml of SFM II medium containing 4 mM L-glutamine, 10 μg/ml Vitamin K1 and 200 μg/ml Geneticin. The media chamber was filled with 1 L of the same medium. The CELLine cultures were seeded at >1.7×10⁶ cells/ml, incubated for three to seven days and harvested. After the growth period, the cell chambers were re-seeded with approximately 3 ml of the harvested cells, and additional media up to 15-19 ml. The media chambers were emptied and refilled with 1 L of fresh medium at each harvest. The growth/harvest cycle was repeated up to six or more times, after which the cultures were discarded. The harvested cell supernatants were adjusted to 5 mM EDTA and 2 mM benzamidine, and filtered through a 2.0 um Millex AP 20 filter prior to storage at −70° C.

### Example 3

### Purification of Factor IX Recombinant Proteins

Factor IX proteins were purified to homogeneity (as determined by SDS-PAGE) from conditioned medium by the following steps. The filtered medium was applied to a High Q Macro-Prep anion-exchange column (Bio-Rad). The column was equilibrated prior to loading and washed extensively with Tris buffer (25 mM Tris, 200 mM NaCl, 1 mM benzamidine, 0.01% (w/w) NaN₃ (pH 7.4)). The column was eluted over a linear gradient of 0-10 mM CaCl₂. Eluted protein was concentrated by centrifugation filtration (Millipore Ultracel, Mr 30,000 cutoff) and the concentrates adjusted to 10 mM EDTA. The concentrated proteins were applied to a Mono Q HR5/5 anion-exchange column (Amersham Biosciences). The column was equilibrated and washed extensively with Tris buffer (25 mM Tris, 25 mM NaCl (pH 7.4)). Proteins were eluted with a linear gradient of 25 mM to 1.0 M NaCl over 20 minutes (flow rate of 1.0 ml/min) Eluted protein was concentrated by centrifugation filtration and adjusted to 100 mM NaCl. Protein concentrations were determined by colorimetric assay (BCA protein assay, Pierce).

### Example 4

### Binding of Mutant Factor IX Molecules to Membranes

Factor IX interaction with membrane vesicles was determined at a composition of PS/PC (5/95) and at 5 mM calcium in 0.05 M Tris buffer, pH 7.5 containing 0.1 M NaCl. Light scattering was monitored at 320 nm in a fluorescence spectrophotometer by methods previously described (Nelsestuen and Lim, Biochemistry, 16:4164-4171, 1977). Bound protein was determined from the relationship:

I₂/I₁=(M₂/M₁)²(∂n₂/∂c₂/∂n₁/∂c₁)²

where I₂/I₁ is the ratio of light scattering intensity of the protein-vesicle complex to that of the vesicles alone, M₂/M₁ is the molecular weight ratio of the complex to that of the vesicles alone, and ∂n/∂c is the refractive index increment for the two species. All intensities were corrected for background from buffer and added protein.

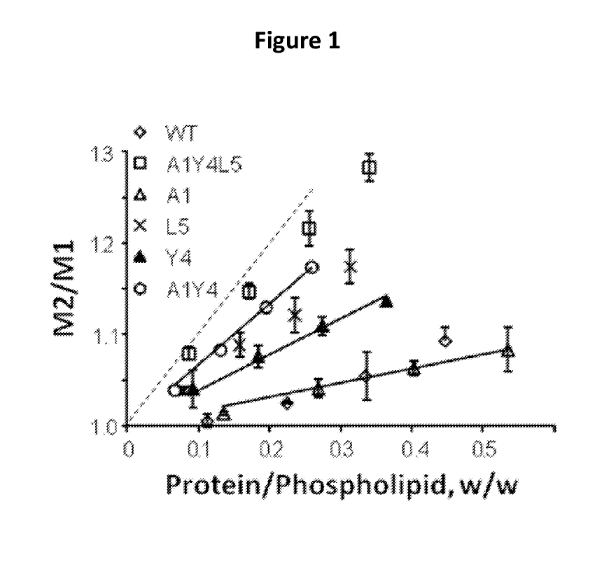

Binding measurements were determined in triplicate, and the results are illustrated in FIG. 1. The dashed line indicates the theoretical limit if all of the added protein bound to the membrane. It was apparent that the triple mutant approached this limit, while the WT and Y1A variant had similar and low affinity binding. The Y4 and L5 variants, as well as combinations, showed intermediate affinity. The resulting binding constants for the different proteins are given in Table 2. Dissociation constants were calculated from the equation:

K_{D}=[protein_{free}][phospholipid_{free}]/[protein-phospholipid complex]

[Protein_{free]} was obtained from the difference between total added protein and free, [protein-phospholipid complex], [phospholipid_{free}] was determined by the difference between the concentration of total protein binding sites (assuming maximum binding capacity of 1/1 (w/w) for factor IX-phospholipid) and [protein-phospholipid complex] was determined from light scattering change and the total phospholipid concentration.

TABLE 2
Binding constants for Factor IX proteins and variants

| Factor IX | K_{D} | Fold change | Theoretical change (from | |
|---|---|---|---|---|
| protein | (μM) | from WT | values for single sites) | Synergy |
| WT | 2.66 | 1.0 | | |

Single site changes

| | | |
|---|---|---|
| Y1A | 2.68 | 1.0 |
| G4Y | 1.06 | 2.5 |
| K5L | 0.40 | 6.7 |

Multiple site changes

| | | | | |
|---|---|---|---|---|
| Y1A/G4Y | 0.62 | 4.3 | 2.5 | 1.7 |
| Y1A/G4Y/K5L | 0.10 | 27 | 17 | 1.6 |

The results shown in Table 2 indicate that a combination of sites was more effective than the sum of the individual sites. This synergy was not anticipated since, for example, prior studies of factor VIIa suggested that all site enhancements were independent of each other (Harvey et al., J. Biol. Chem., 278:8363-8369, 2003).

### Example 5

### Activation of Recombinant Factor IX Proteins by Factor VIIa:Tissue Factor

To demonstrate the impact of increased membrane binding affinity on factor IX activation, the factor IX variant with three substitutions (Y1A/G4Y/K5L) was activated by incubation with Tissue factor- (TF-) containing vesicles and factor VIIa. TF-vesicles were formed using published methods (Stone et al., supra) using full length TF (American Diagnostica) and purified phospholipids. The appropriate amounts of phospholipids were dried from organic solvent to form a film on a glass tube, and were suspended in solvent containing octylglucoside. TF was added to the appropriate level and the solution was dialyzed for at least 72 hours to remove detergent. The resulting TF-vesicles containing phosphatidylserine (PS)/phosphatidylcholine (PC) (10:90) were added to the reaction mixture to a final concentration of 27 μg/mL with 2.6 nM tissue factor in Tris buffer. Recombinant WT or mutant factor IX was added to a final concentration of 180 nM, along with recombinant factor VIIa (6.9 nM). Reactions were allowed to proceed in buffer (0.05 M Tris, pH 7.5, 0.1 M NaCl, 1 mM CaCl, 1 mg/mL bovine serum albumin) for 10 minutes. Reactions were stopped by addition of ethanol to 36% (v/v), and factor IXa substrate (Biophen CS51(09), Hyphen Biomed) was added. The change in absorbance at 405 nm was monitored for 10 minutes in a DU-70 spectrophotometer. Appropriate background values were obtained from reactions lacking either factor IX or phospholipid-TF. The net (background subtracted) absorbance change was 0.0153/min for the variant vs. 0.0016/min for recombinant WT factor IX. The enhancement of function for the variant was 9.5-fold.

The reactions also were run with the same components as described above, but with 0.4 times as much factor IX protein (72 nM). The net absorbance change at 405 nm after a 10-minute activation was 0.0082/min for the variant vs. 0.0009/min for recombinant WT factor IX, indicating a 9-fold enhanced function.

The variant also was compared using membrane vesicles containing PS/PC (1/99) with TF. After activation, the variant gave a net absorbance change of 0.0063/min, while recombinant WT gave a net change of 0.0005/min, a 12-fold enhancement. In general, the difference between WT and variant proteins increased with lower PS content.

### Example 6

### Blood Coagulation in Factor IX-Deficient Plasma

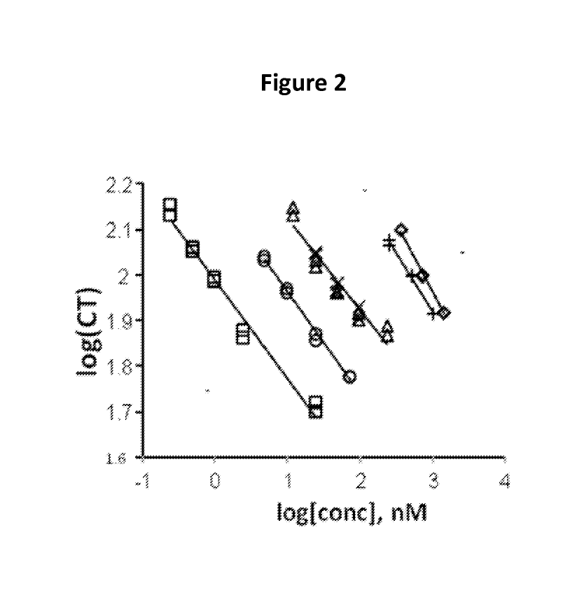

Recombinant factor IX-WT (36 μM) and factor IX (Y1A/G4Y/K5L) (22.9 μM) were independently activated by addition of factor XIa (Enzyme Research Laboratory, 1:100 to factor IX) in Tris buffered saline, pH 7.3. Preparations were incubated at 37° C. for one hour and then overnight at room temperature. Full activation was determined by gel electrophoresis to detect the appropriate cleavage with no remaining factor IX. Factor IX-deficient plasma that was anticoagulated with Na Citrate was warmed to 37° C. Coagulation times were determined by the hand tilt method for a mixture of 40 μL of plasma with phospholipid (PS/PC 25/75, 67 μg/mL final concentration) in 0.10 mL of Tris Buffer pH 7.35, 0.1 M NaCl containing 7.5 mM CaCl₂ and the factor IXa or VIIa. Assays without added factors did not clot within 180 seconds. Data were plotted as the log(clotting time) vs. log(factor IXa), with the results shown in FIG. 2. Under these conditions, the factor IXa variant showed approximately 8-fold higher activity than WT IXa.

Other conditions also were tested. As expected for proteins with higher membrane binding affinity, the enhancement was increased at low CaCl₂ concentration and with membranes of lower PS content. For example, the use of Tris buffer with 3.75 mM CaCl₂ and membranes of 10% PS gave a 15-fold difference between the variant and WT. Clotting time also was sensitive to phospholipid concentration. Thus, the relative difference between the mutant and WT factor IXa remained similar but varied with Ca concentration and membrane composition, in a manner similar to that presented in Example 5.

In a separate experiment, a standard APTT reagent was used as the phospholipid source. In this case, the variant gave only 2-fold higher activity than WT protein when analyzed by the methods shown in FIG. 2. This was consistent with a membrane composition of the APTT test that contained very high levels of PS or other components that enhance membrane affinity. For very high affinity membranes, WT protein will bind to a high extent, with less benefit of the variant.

### Example 7

### Factor IXa as a Potential Bypassing Agent

Factor IXa variants were tested with factor VIII-deficient plasma. This illustrated the potential use of factor IXa as a bypass agent in cases of factor VIII-deficiency. In this case, coagulation was dependent on factor VIII-independent activity of factor IXa. The IXa is presumed to bind to the membrane and activate membrane-bound factor X to Xa or to directly activate prothrombin to thrombin.

The triple mutant showed approximately 18-fold enhanced function relative to WT factor IXa (FIG. 2). The membranes contained PS/PC (25/75) and were present at 67 μg/mL. Other conditions included 7.5 mM calcium in Tris buffer.

It is important to note that the activity of mutant factor IXa in factor VIII-deficient plasma was 16% that of WT factor IXa in factor VIII-competent plasma. This level of activity may be sufficient to allow mutant factor IX zymogen to act as a therapeutic in factor VIII-deficient patients. That is, the factor IXa activation pathways in Hemophilia A are presumed to be normal. Generation of factor IXa with 16% activity in the absence of factor VIII may be sufficient to mediate proper blood coagulation. Increased activation of factor IX by tissue factor-factor VIIa (Example 5) would increase functional coagulation even further.

Recombinant factor VIIa has been used as a bypass agent. Results for factor VIIa protein (NOVOSEVEN®, Novo Nordisk Inc., Princeton, N.J.) under the same assay conditions are shown in FIG. 2. NOVOSEVEN® was 1.5-fold more effective than WT factor IXa. However, the triple mutant of factor IX was 12-fold more effective than NOVOSEVEN®. Thus, the factor IXa variant would provide enhanced function in factor VIII-deficient blood, providing a possible mechanism for bypass in cases of factor VIII-deficiency with inhibitors. Conditions of lower CaCl₂ concentration and 10% PS membranes showed up to a 30-fold difference between the variant and WT factor IXa. The maximum benefit of the variant approximately equaled its advantage in membrane binding.

### Example 8

### Factor IXa and IX in Whole Blood Clotting Tests

An important test of proteins with elevated membrane affinity is in true biological membranes. Many commercial in vitro coagulation tests contain proprietary phospholipid compositions that are of high affinity, quite unlike that of biological membranes. As shown for factor VIIa, use of these commercial membrane compositions can entirely conceal the benefit of high membrane affinity mutants (Nelsestuen et al., supra). Thus, a valuable assay is the whole blood clotting test that depends entirely on biological membranes found in whole blood. Coagulation tests were conducted using an ACT-LR cuvette in a HEMOCHRON® Jr. Signature microcoagulation apparatus (International Technidyne Corp., Edison, N.J.). This clotting test has an automatic cut-off at 400 seconds. Blood containing 0.5% of normal factor IX or VIII levels gives a measureable clotting time (Henderson et al., Thromb. Haemost., 88:89-103, 2002).

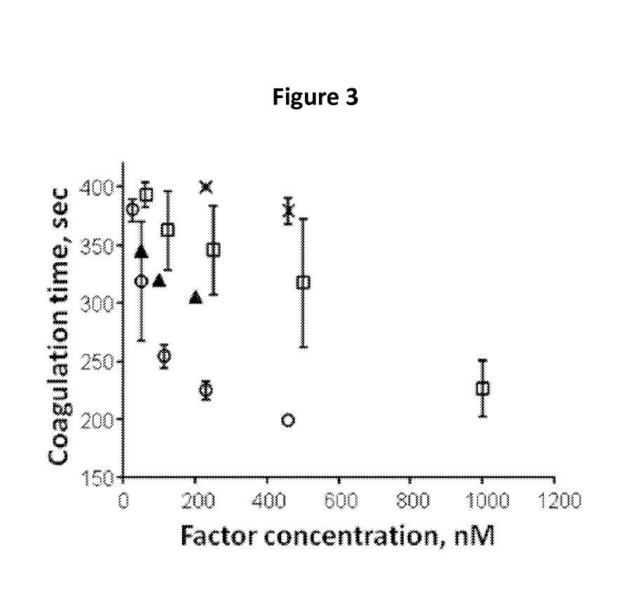

FIG. 3 shows the results for factor IXa and factor IX proteins. The triple mutant factor IXa was 5-10 times more active than WT factor IXa, and was approximately equal in activity to NOVOSEVEN® but had a much greater ability to lower blood clotting times. The zymogen triple mutant factor IX gave a measureable clotting time at 460 nM (FIG. 3). WT zymogen did not give a measureable clotting time at a concentration of 1000 nM.

The whole blood clotting test is dependent on intrinsic activation of factor IX by factor XIa. This pathway does not involve membrane association, so the variant would not benefit from this aspect of its increased function. In vivo activation by TF-factor VIIa does involve membrane association. Consequently, the variant should show more than 10-fold enhancement under in vivo conditions.

### Example 9

### Further Enhancement Through Additional Modifications

Factor IX with high membrane-binding affinity was further modified by additional site-directed mutations. Specifically, factor IX zymogens were generated to contain the triple mutant Gla domain (Y1A/G4Y/K5L) in combination with mutations at other sites that may enhance protein function (Hartman et al., supra). Site directed changes were generated as described in Example 1. Cells were transfected with the relevant vectors and cell growth conducted as described in Example 2. Proteins were purified from conditioned medium as described in Example 3. Factor IX was converted to factor IXa by the action of factor XIa as described in Example 6. Activity of the factor IXa proteins was measured in the whole blood clotting test described in Example 8.

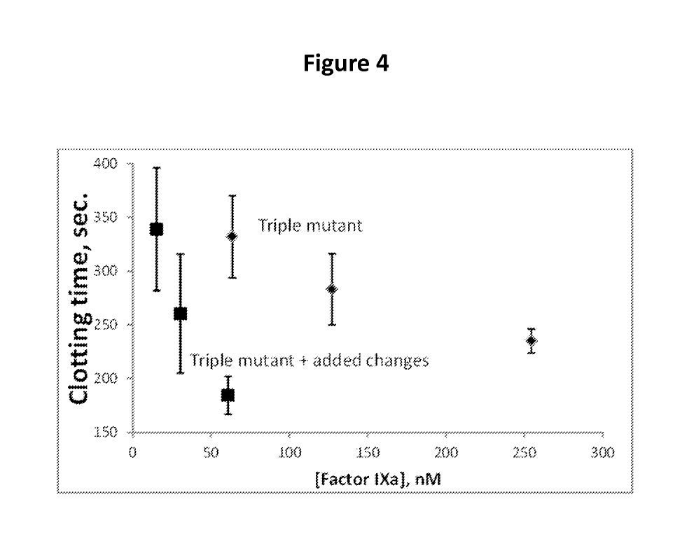

As shown in FIG. 4, the maximum activity was achieved by additional of three additional site changes K265T/I383V/E388G; the maximum activity of the full modification was about 5-times the activity of the triple mutant.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A variant factor IX or factor IXa polypeptide having enhanced membrane binding affinity relative to a corresponding native factor IX or factor IXa polypeptide, wherein the polypeptide comprises a modified Gla domain with an amino acid substitution at one or more of positions 1, 4, and 5 as compared to the sequence set forth in SEQ ID NO:1 or SEQ ID NO:2.

2. The polypeptide of claim 1, wherein the Gla domain comprises an amino acid substitution at position 1.

3. The polypeptide of claim 2, wherein an alanine residue is substituted at position 1.

4. The polypeptide of claim 1, wherein the Gla domain comprises an amino acid substitution at position 4.

5. The polypeptide of claim 4, wherein a tyrosine residue is substituted at position 4.

6. The polypeptide of claim 1, wherein the Gla domain comprises an amino acid substitution at position 5.

7. The polypeptide of claim 6, wherein a leucine residue is substituted at position 5.

8. The polypeptide of claim 1, wherein the Gla domain comprises amino acid substitutions at positions 1 and 4.

9. The polypeptide of claim 8, wherein an alanine residue is substituted at position 1 and a tyrosine residue is substituted at position 4.

10. The polypeptide of claim 1, wherein the Gla domain comprises amino acid substitutions at positions 1, 4, and 5.

11. The polypeptide of claim 10, wherein an alanine residue is substituted at position 1, a tyrosine residue is substituted at position 4, and a leucine residue is substituted at position 5.

12. A pharmaceutical composition comprising a pharmaceutically acceptable carrier and an amount of a variant factor IX or factor IXa polypeptide effective to increase clot formation in a mammal, wherein the polypeptide comprises a modified Gla domain with an amino acid substitution at one or more of positions 1, 4, or 5 as compared to the sequence set forth in SEQ ID NO:1 or SEQ ID NO:2.

13. The pharmaceutical composition of claim 12, wherein the polypeptide comprises a modified Gla domain with amino acid substitutions at positions 1, 4, and 5.

14. The pharmaceutical composition of claim 13, wherein an alanine residue is substituted at position 1, a tyrosine residue is substituted at position 4, and a leucine residue is substituted at position 5.

15. A mammalian host cell that expresses a variant factor IX or factor IXa polypeptide having enhanced membrane binding affinity relative to a corresponding native factor IX or factor IXa polypeptide, wherein the polypeptide comprises a modified Gla domain with an amino acid substitution at one or more of positions 1, 4, or 5 as compared to the sequence set forth in SEQ ID NO:1 or SEQ ID NO:2.

16. The host cell of claim 15, wherein the polypeptide comprises a modified Gla domain with amino acid substitutions at positions 1, 4, and 5.

17. The host cell of claim 15, wherein an alanine residue is substituted at position 1, a tyrosine residue is substituted at position 4, and a leucine residue is substituted at position 5.

18. A method of increasing clot formation in a mammal, comprising administering to the mammal an amount of a variant factor IX or factor IXa polypeptide effective to increase clot formation in the mammal, wherein the polypeptide comprises a modified Gla domain with an amino acid substitution at one or more of positions 1, 4, or 5 as compared to the sequence set forth in SEQ ID NO:1 or SEQ ID NO:2.

19. The method of claim 18, wherein the polypeptide comprises a modified Gla domain with amino acid substitutions at positions 1, 4, and 5.

20. The method of claim 18, wherein an alanine residue is substituted at position 1, a tyrosine residue is substituted at position 4, and a leucine residue is substituted at position 5.

21. An isolated nucleic acid comprising a nucleic acid sequence encoding a variant factor IX or factor IXa polypeptide having enhanced membrane binding affinity relative to a corresponding native factor IX or factor IXa polypeptide, wherein the polypeptide comprises a modified Gla domain with an amino acid substitution at one or more of positions 1, 4, or 5 as compared to the sequence set forth in SEQ ID NO:1 or SEQ ID NO:2.

22. The nucleic acid of claim 21, wherein the polypeptide comprises a modified Gla domain with amino acid substitutions at positions 1, 4, and 5.

23. The nucleic acid of claim 21, wherein an alanine residue is substituted at position 1, a tyrosine residue is substituted at position 4, and a leucine residue is substituted at position 5.

24. A method for producing a variant factor IX or factor IXa polypeptide having enhanced membrane binding affinity relative to a corresponding native factor IX or factor IXa polypeptide, wherein the polypeptide comprises a modified Gla domain with an amino acid substitution at one or more of positions 1, 4, or 5 as compared to the sequence set forth in SEQ ID NO:1 or SEQ ID NO:2, the method comprising:
(a) providing a culture of the mammalian host cell of claim 15 under conditions that permit expression of the polypeptide, and
(b) recovering the polypeptide.

25. The method of claim 24, wherein the polypeptide comprises a modified Gla domain with amino acid substitutions at positions 1, 4, and 5.

26. The method of claim 24, wherein an alanine residue is substituted at position 1, a tyrosine residue is substituted at position 4, and a leucine residue is substituted at position 5.
